(19) United States
(12) Patent Application Publication
(10) Pub. No.: US 2026/0174337 A1
(43) Pub. Date: Jun. 25, 2026

(54) VR APPARATUS

(71) Applicant: Murata Manufacturing Co., Ltd., Nagaokakyo-shi (JP)

(72) Inventor: Kenjiro OKAGUCHI, Nagaokakyo-shi (JP)

(21) Appl. No.: 19/541,465

(22) Filed: Feb. 17, 2026

Related U.S. Application Data
(63) Continuation of application No. PCT/JP2024/029236, filed on Aug. 19, 2024.

(30) Foreign Application Priority Data
Sep. 4, 2023 (JP) 2023-142813

(51) Int. Cl. A61B 5/01

(52) U.S. Cl. CPC A61B 5/01

(57) ABSTRACT

A VR apparatus includes a housing, air blowing devices, speakers, and a VR control module. The housing has a first opening capable of communicating with an ear canal opening of a user. The air blowing devices are provided in the housing and generate airflow to be delivered from the first opening to the outside of the housing. The speakers are provided in the housing and generate sound to be delivered from the first opening to the outside of the housing. The VR control module is configured to, on the basis of virtual reality provided to the user, output an air blowing control signal to the air blowing devices and output a sound radiation control signal to the speakers in such a manner as to synchronize a generation timing of the sound and a generation timing of the airflow in time.

### CROSS REFERENCE TO RELATED APPLICATION

This is a continuation of International Application No. PCT/JP2024/029236 filed on August 19, 2024 which claims priority from Japanese Patent Application No. 2023-142813 filed on September 4, 2023. The contents of these applications are incorporated herein by reference in their entireties.

### BACKGROUND OF THE DISCLOSURE

### Field of the Disclosure

The present disclosure relates to a VR apparatus that is wearable on a user’s head.

### Description of the Related Art

A simulation system is described in Japanese Unexamined Patent Application Publication No. 2018-126341. The simulation system of Japanese Unexamined Patent Application Publication No. 2018-126341 enables a user to experience virtual reality by using a head-mounted display apparatus and an air blower.

The head-mounted display apparatus is worn on the user’s head. The air blower is disposed at a position separated by a predetermined distance in front of the user. The head-mounted display apparatus provides the user with video and sound. The air blower delivers airflow toward the user.

### BRIEF SUMMARY OF THE DISCLOSURE

However, in conventional configurations including that of Japanese Unexamined Patent Application Publication No. 2018-126341, an apparatus that provides virtual reality with which a high sense of immersion can be obtained becomes large in size. Thus, it is difficult for the user to readily experience virtual reality with which a high sense of immersion can be obtained.

Accordingly, a possible benefit of the present disclosure is to provide a VR apparatus (virtual reality reproduction apparatus) that can implement, with simple equipment, virtual reality with which a high sense of immersion can be obtained for a user.

A VR apparatus of this disclosure includes a housing, an air blowing device, a speaker, and a VR control module. The housing has a first opening capable of communicating with an ear canal opening of a user. The air blowing device is provided in the housing and generates airflow to be delivered from the first opening to the outside of the housing. The speaker is provided in the housing and generates sound to be delivered from the first opening to the outside of the housing. The VR control module is configured to, on the basis of virtual reality provided to the user, output an air blowing control signal to the air blowing device and output a sound radiation control signal to the speaker in such a manner as to synchronize a generation timing of the sound and a generation timing of the airflow in time.

With this configuration, the user can experience the sound and the airflow based on the virtual reality simply by wearing the VR apparatus on the head. Thus, the virtual reality can be reproduced with simple equipment such that a high sense of immersion is obtained compared with a case in which only video or only sound is provided.

According to this disclosure, it is possible to implement, with simple equipment, virtual reality with which a high sense of immersion can be obtained for a user.

### BRIEF DESCRIPTION OF THE SEVERAL VIEWS OF THE DRAWINGS

FIG. 1 is a functional block diagram of a VR apparatus according to a first embodiment.

FIG. 2 is a diagram depicting an example of how the VR apparatus according to the first embodiment is worn.

FIG. 3 is a side cross-sectional view depicting an example of a configuration of headphones of the VR apparatus according to the first embodiment.

FIG. 4 is a diagram depicting an example of a state in which sound and airflow are provided by the VR apparatus according to the first embodiment.

FIG. 5 is a side cross-sectional view depicting an example of a configuration of headphones of a VR apparatus according to a second embodiment.

FIG. 6 is a side cross-sectional view depicting an example of a configuration of an air blowing device.

FIG. 7 is a graph depicting an example of acoustic frequency characteristics of a piezoelectric pump and a motor (axial flow fan).

FIG. 8A is a side cross-sectional view depicting an example of a configuration of an earphone of a VR apparatus according to a third embodiment; and FIG. 8B is a side cross-sectional view depicting an example of flows of airflow and sound.

FIG. 9 is a diagram depicting an example of how the VR apparatus according to the third embodiment is worn.

FIG. 10 is a functional block diagram of a VR apparatus according to a fourth embodiment.

FIG. 11 is a side cross-sectional view depicting an example of a configuration of an earphone of the VR apparatus according to the fourth embodiment.

FIG. 12A is a perspective view depicting an example of how the VR apparatus according to the fourth embodiment is worn; and FIG. 12B is an enlarged cross-sectional view of a vicinity of a wearing position.

FIG. 13 is a functional block diagram of a VR apparatus according to a fifth embodiment.

FIG. 14 is a side cross-sectional view depicting an example of a configuration of an earphone of the VR apparatus according to the fifth embodiment.

FIG. 15 is a schematic configuration diagram of a temperature sensor 40E of a VR apparatus according to a sixth embodiment.

FIG. 16 is an enlarged cross-sectional view of a vicinity of a wearing position of the VR apparatus according to the sixth embodiment.

FIG. 17A is a circuit diagram depicting an example of an activation circuit of an air blowing control section; and FIG. 17B depicts an output voltage characteristic of the activation circuit.

FIG. 18 is a circuit diagram depicting an example of a drive circuit of the air blowing control section.

### DETAILED DESCRIPTION OF THE DISCLOSURE

### FIRST EMBODIMENT

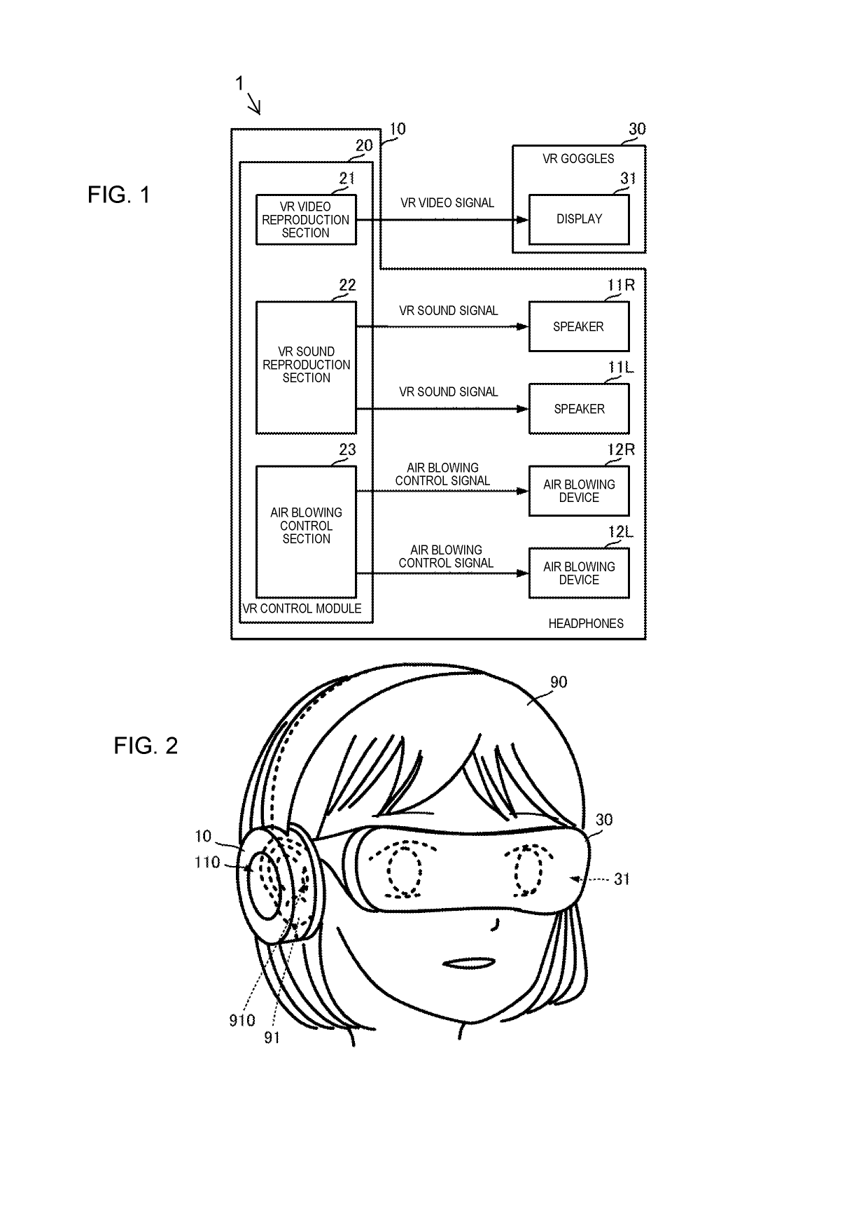

A VR apparatus according to a first embodiment of the present disclosure is described with reference to the drawings. FIG. 1 is a functional block diagram of the VR apparatus according to the first embodiment. FIG. 2 is a diagram depicting an example of how the VR apparatus according to the first embodiment is worn.

### Configuration of Functional Portions of VR Apparatus 1

As depicted in FIG. 1, the VR apparatus 1 includes headphones 10 and VR goggles 30. The headphones 10 include a speaker 11R, a speaker 11L, an air blowing device 12R, an air blowing device 12L, and a VR control module 20. In the present embodiment, a configuration in which the VR control module 20 is included in the headphones 10 is depicted. However, the VR control module 20 may be included in the VR goggles 30, or may be provided separately from the headphones 10 and the VR goggles 30, or in the cloud or the like.

The speaker 11R is for the right channel, and the speaker 11L is for the left channel. The air blowing device 12R is for the right ear, and the air blowing device 12L is for the left ear.

The headphones 10 include a right-ear main body and a left-ear main body. The speaker 11R and the air blowing device 12R are included in the right-ear main body. The speaker 11L and the air blowing device 12L are included in the left-ear main body.

The VR control module 20 includes a VR video reproduction section 21, a VR sound reproduction section 22, and an air blowing control section 23. The VR video reproduction section 21 reproduces a VR video signal and outputs it to a display 31 of the VR goggles 30. The VR sound reproduction section 22 reproduces a VR sound signal and outputs it to the speaker 11R and the speaker 11L. The air blowing control section 23 generates an air blowing control signal and outputs it to the air blowing device 12R and the air blowing device 12L.

The VR video reproduction section 21, the VR sound reproduction section 22, and the air blowing control section 23 output the VR video signal, the VR sound signal, and the air blowing control signal in time synchronization on the basis of virtual reality (VR) provided to a user who uses the VR apparatus 1.

The display 31 displays VR video based on the VR video signal. The speaker 11R and the speaker 11L generate sound based on the VR sound signal. The air blowing device 12R and the air blowing device 12L are driven on the basis of the air blowing control signal to generate airflow.

As depicted in FIG. 2, the headphones 10 of such a VR apparatus 1 are worn on the head of a user 90 and used. The headphones 10 are worn so as to cover both ears of the user 90. The right-ear main body of the headphones 10 is worn on the right ear, and although depiction is omitted, the left-ear main body of the headphones 10 is worn on the left ear. The VR goggles 30 are worn so as to cover the eyes of the user 90.

This allows the user 90 to view the VR video by the display 31 of the VR goggles 30. Further, by the headphones 10, the user 90 can hear the sound with both ears and feel the airflow with both ears.

In this manner, the VR apparatus 1 can provide the user with the airflow in addition to the video and the sound. Further, the VR apparatus 1 can provide the airflow in time synchronization, in addition to the time synchronization of the video and the sound. That is, the VR apparatus 1 can cause the user to experience VR not only through the visual sense and the auditory sense of the user but also through the tactile sense thereof. Thus, the VR apparatus 1 can provide a high sense of immersion to the user.

For example, the VR control module 20 outputs the air blowing control signal in time synchronization with the VR video signal and the VR sound signal at, in virtual reality, a timing of providing cool-feeling video and sound to the user, a timing of providing video and sound that give a surprise to the user, or a timing of providing video and sound that cause the user to have a sense of fear. This allows the user to experience coolness, surprise, or fear by not only the video and the sound but also airflow.

In this case, because the speaker 11R, the speaker 11L, the air blowing device 12R, and the air blowing device 12L are included in the headphones 10, the VR apparatus 1 can be made compact. Thus, the user 90 can readily experience virtual reality with a high sense of immersion with simple equipment.

Moreover, because the speaker 11R, the speaker 11L, the air blowing device 12R, and the air blowing device 12L are included in the headphones 10, the sound radiation sources and the air blowing sources are close to the ears (user 90). Accordingly, a time difference between the arrival of the sound at the ears and the arrival of the airflow at the ears is unlikely to occur. Thus, the VR apparatus 1 can provide a higher sense of immersion to the user 90.

### Structural Example of Headphones 10

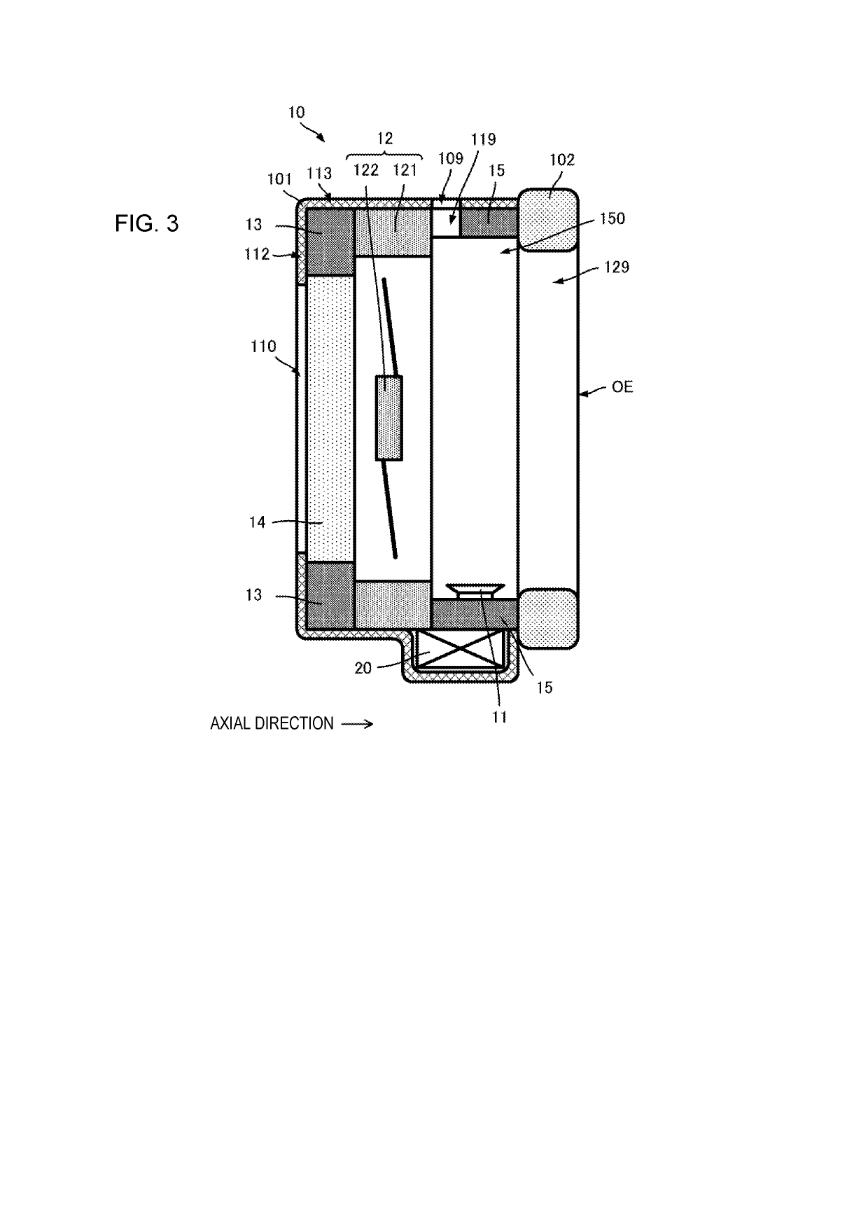

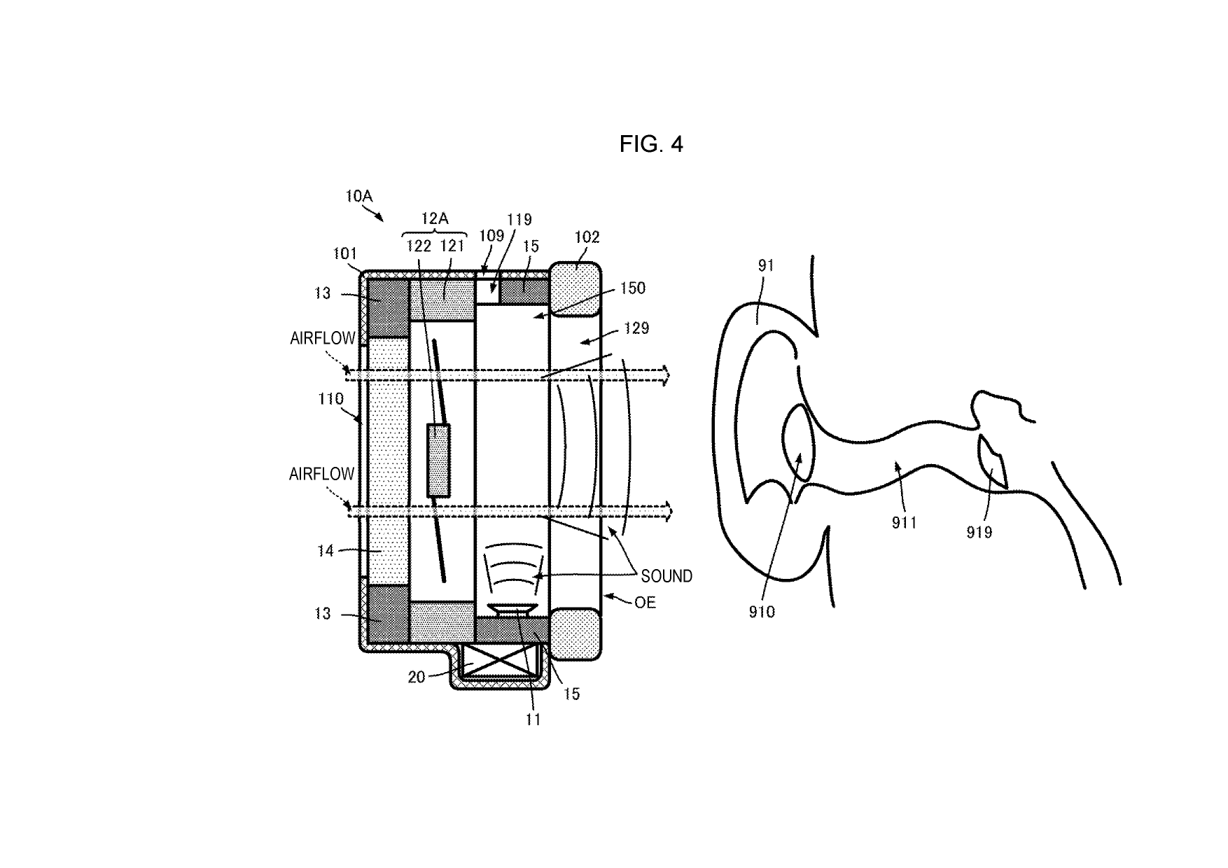

To implement the above functions, the headphones 10 include, for example, a configuration depicted in FIG. 3. FIG. 3 is a side cross-sectional view depicting an example of a configuration of the headphones of the VR apparatus according to the first embodiment. FIG. 4 is a diagram depicting an example of a state in which sound and airflow are provided by the VR apparatus according to the first embodiment. In FIGS. 3 and 4, the right-ear main body of the headphones 10 is depicted as an example. However, depiction and description of the left-ear main body are omitted because it has the same structure as the right-ear main body. Further, hereinafter, the right-ear main body of the headphones 10 is simply referred to as the headphones 10.

As depicted in FIG. 3, the headphones 10 include a housing 101, a speaker 11, an air blowing device 12, a first sound absorbing member 13, a second sound absorbing member 14, a holding member 15, and an ear pad 102. The fixing member of the disclosure of the present application is configured by the first sound absorbing member 13 and the second sound absorbing member 14.

The housing 101 has a cylindrical shape having a bottom wall 112 and a side wall 113. The housing 101 is open at an end portion on the side opposite to the bottom wall 112 in the axial direction of the cylindrical shape. An opening 110 having a predetermined opening area is formed in the bottom wall 112. The housing 101 is composed of a material having rigidity that allows the shape to be kept constant, and is composed of, for example, a resin, a metal, or the like.

The ear pad 102 has an annular shape. The ear pad 102 is composed of a material having cushioning properties.

The ear pad 102 has a central space 129. The ear pad 102 is attached to the housing 101 such that the central space 129 communicates with the opening of the housing 101. The opening surface of the ear pad 102 on the side opposite to the mounting surface to the housing 101 is an opening OE of the headphones 10. The opening OE serves as a sound radiation surface and an airflow delivery surface as the headphones 10.

The air blowing device 12 is configured with an axial flow fan. The air blowing device 12 includes a housing 121 and a fan 122. The air blowing device 12 is disposed at an intermediate position in the axial direction of the housing 101. The air blowing device 12 is disposed such that an air blowing surface thereof is substantially orthogonal to the axial direction of the housing 121. An outer peripheral surface of the housing 121 abuts against an inner peripheral surface of the side wall 113 of the housing 101.

The first sound absorbing member 13 is composed of a material having low air permeability and high sound absorbing properties. The first sound absorbing member 13 has an annular shape.

The first sound absorbing member 13 is disposed on the bottom wall 112 side of the housing 101 relative to the air blowing device 12. In other words, the first sound absorbing member 13 is disposed on the side opposite to the opening OE with the air blowing device 12 being a reference in the housing 101.

The first sound absorbing member 13 abuts against the bottom wall 112 and the side wall 113. A surface of the first sound absorbing member 13 on the side opposite to the bottom wall 112 abuts against the housing 121 of the air blowing device 12.

The second sound absorbing member 14 is disposed in a through-hole penetrating the first sound absorbing member 13. The second sound absorbing member 14 overlaps the opening 110 of the bottom wall 112 of the headphones 10.

The second sound absorbing member 14 is composed of a material having high air permeability and sound absorbing properties. The second sound absorbing member 14 is composed of, for example, glass wool, steel wool, or the like.

By including the first sound absorbing member 13 and the second sound absorbing member 14, the headphones 10 can suppress leakage of undesired sound caused by vibration of the axial flow fan of the air blowing device 12, axial misalignment thereof, or the like to the outside of the housing 101. Further, by including the second sound absorbing member 14, the headphones 10 can ensure air supply to the air blowing device 12 while suppressing leakage of the above undesired sound.

That is, the first sound absorbing member 13 primarily implements absorption of undesired sound, and the second sound absorbing member 14 ensures air supply to the air blowing device 12 while assisting in absorbing undesired sound.

The holding member 15 has an annular shape and is composed of a material having predetermined rigidity. The holding member 15 may be composed of a material having low sound reflectivity.

The holding member 15 abuts against the inner peripheral surface of the side wall 113 of the housing 101. The holding member 15 is disposed between the air blowing device 12 and the ear pad 102 in the axial direction of the housing 101. An auxiliary air-discharge hole 119 is formed in the holding member 15. The auxiliary air-discharge hole 119 communicates with the outside of the housing 101 through a through-hole 109 formed in the side wall 113 of the housing 101.

The auxiliary air-discharge hole 119 and the through-hole 109 may be omitted. Including the auxiliary air-discharge hole 119 and the through-hole 109 makes it possible to suppress the occurrence of a situation in which, when the headphones 10 are worn by the user 90, the inside of the headphones 10 becomes undesirably high in pressure to cause discomfort to the user 90.

The speaker 11 is fixed to the holding member 15. Specifically, the speaker 11 is disposed such that a sound radiation surface thereof faces a central space of the holding member 15 and is exposed to this central space.

A main sound radiation direction of the speaker 11 is orthogonal to the axial direction of the housing 101. The manner in which the speaker 11 is disposed is not limited thereto.

The shape of the speaker 11 is not limited, but the speaker 11 may be as small as possible.

With such a configuration, as depicted in FIG. 4, the headphones 10 can provide sound and airflow from the opening OE to an ear 91 of the user 90. The sound and the airflow enter an external auditory canal 911 through an ear canal opening 910. The sound and the airflow that have entered the external auditory canal 911 reach an eardrum 919.

Thus, the user 90 can hear the sound and can also experience the sensation of the airflow almost simultaneously. Accordingly, the VR apparatus 1 can provide a high sense of immersion to the user 90.

In particular, the vicinity of the eardrum 919 is at a temperature close to the core temperature of a person. Accordingly, the VR apparatus 1 can provide a higher cooling effect by airflow than when airflow is applied to a region where skin is exposed, such as an arm, a face, or a leg. Further, because the VR apparatus 1 applies airflow to the user 90 from a position very close to the user 90, a high cooling effect can be obtained even with a low airflow volume.

Thus, the VR apparatus 1 can efficiently provide a high sense of immersion to the user 90. Accordingly, the VR apparatus 1 can achieve, for example, reduced power consumption.

### SECOND EMBODIMENT

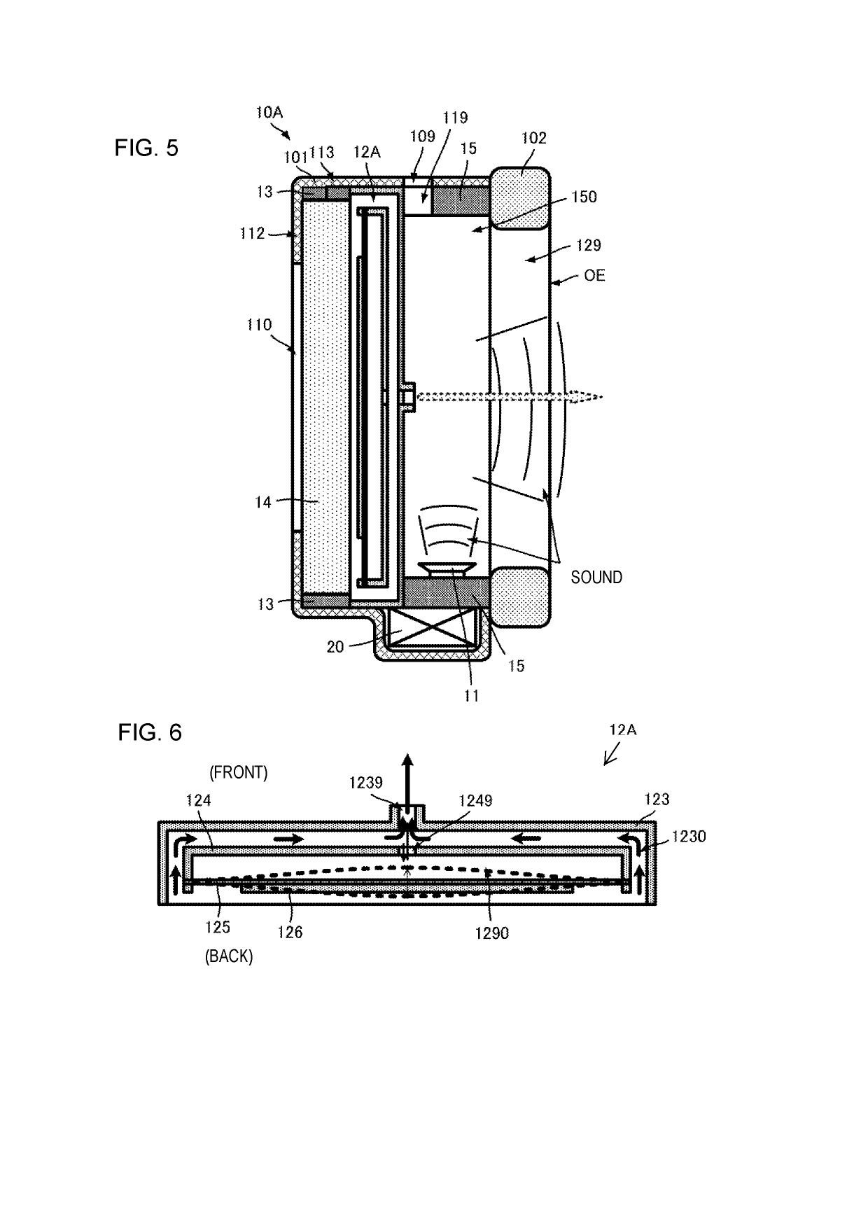

A VR apparatus according to a second embodiment of the present disclosure is described with reference to the drawings. FIG. 5 is a side cross-sectional view depicting an example of a configuration of headphones of the VR apparatus according to the second embodiment.

As depicted in FIG. 5, a VR apparatus 1A according to the second embodiment is different from the VR apparatus 1 according to the first embodiment in an air blowing device 12A. The other configuration of the VR apparatus 1A is the same as that of the VR apparatus 1, and description of the same portions is omitted.

The VR apparatus 1A includes the air blowing device 12A. FIG. 6 is a side cross-sectional view depicting an example of a configuration of the air blowing device.

The air blowing device 12A is a piezoelectric pump. As depicted in FIG. 6, the air blowing device 12A includes an outer housing 123, an inner housing 124, a diaphragm 125, and a piezoelectric element 126.

The outer housing 123 has a rectangular shape in a front view, and has a discharge port 1239 penetrating a front wall at the center of the front wall. The back side of the outer housing 123 is open.

The inner housing 124 has a rectangular shape in a front view, and has a through-hole 1249 penetrating a front wall at the center of the front wall. The back side of the inner housing 124 is open. The shape of the inner housing 124 is smaller than that of the outer housing 123, and is approximately similar thereto.

The inner housing 124 is disposed in an internal space of the outer housing 123. The front direction of the inner housing 124 is the same as that of the outer housing. A flow passage 1230 is formed by a space surrounded by the outer housing 123 and the inner housing 124. When the air blowing device 12A is viewed from the front, the discharge port 1239 of the outer housing 123 and the through-hole 1249 of the inner housing 124 overlap each other.

The diaphragm 125 as a flat plate is disposed on the back side of the inner housing 124. The diaphragm 125 closes the opening of the inner housing 124 on the back side. Thus, in the air blowing device 12A, a pump chamber 1290 surrounded by the inner housing 124 and the diaphragm 125 is formed.

The piezoelectric element 126 is disposed on the diaphragm 125.

By applying a drive signal of a predetermined frequency (drive frequency) to the piezoelectric element 126, the piezoelectric element 126 is deformed and the diaphragm 125 vibrates. Thus, the air blowing device 12A changes the volume of the pump chamber 1290. The air blowing device 12A utilizes this volume change to draw air from the back side of the air blowing device 12A into the pump chamber 1290 through the flow passage 1230 and the through-hole 1249. Further, when discharging air from the pump chamber 1290 through the through-hole 1249, the air blowing device 12A entrains air flowing in from the flow passage 1230 and discharges the air from the discharge port 1239.

Thus, the air blowing device 12A has high directivity in the front direction, and discharges a gas at a predetermined flow velocity.

The air blowing device 12A is disposed such that the front thereof is oriented toward the opening OE. Further, the air blowing device 12A is disposed at such a position that the opening OE and the discharge port 1239 overlap each other when the opening OE is viewed from the outer side.

Thus, headphones 10A of the VR apparatus 1A can achieve operation and effects similar to those of the headphones 10 of the VR apparatus 1. Further, by using the air blowing device 12A configured with the piezoelectric pump, the headphones 10A of the VR apparatus 1A can deliver airflow to the ear canal opening 910 of the user 90 efficiently and effectively compared with the air blowing device 12 configured with the axial flow fan.

Moreover, by using the air blowing device 12A configured with the piezoelectric pump, the headphones 10A of the VR apparatus 1A can suppress noise perceived by the user 90.

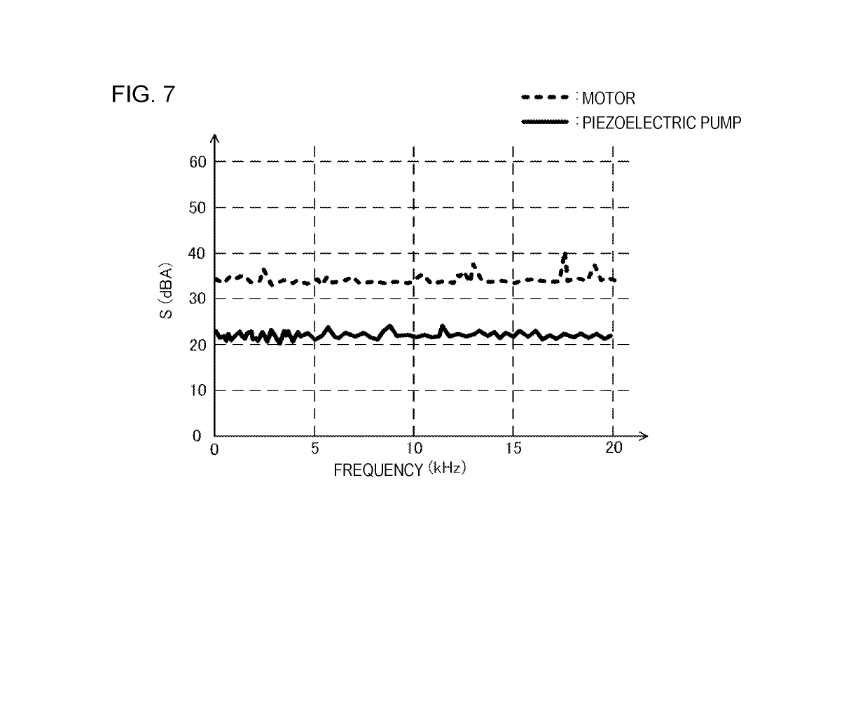

FIG. 7 is a graph depicting an example of acoustic frequency characteristics of the piezoelectric pump and a motor (axial flow fan). FIG. 7 indicates the audible range. In FIG. 7, the horizontal axis indicates the frequency and the vertical axis indicates the sound level, with a solid line representing the piezoelectric pump and a dotted line representing the motor (axial flow fan).

As depicted in FIG. 7, the use of the piezoelectric pump can suppress the sound level in the audible range.

As described above, the headphones 10A of the VR apparatus 1A can suppress noise perceived by the user 90.

Further, the piezoelectric pump is smaller, thinner, and lighter than the axial flow fan. Accordingly, the headphones 10A can achieve further size reduction, and the load on the user 90 when the user 90 wears the headphones 10A can be reduced.

### THIRD EMBODIMENT

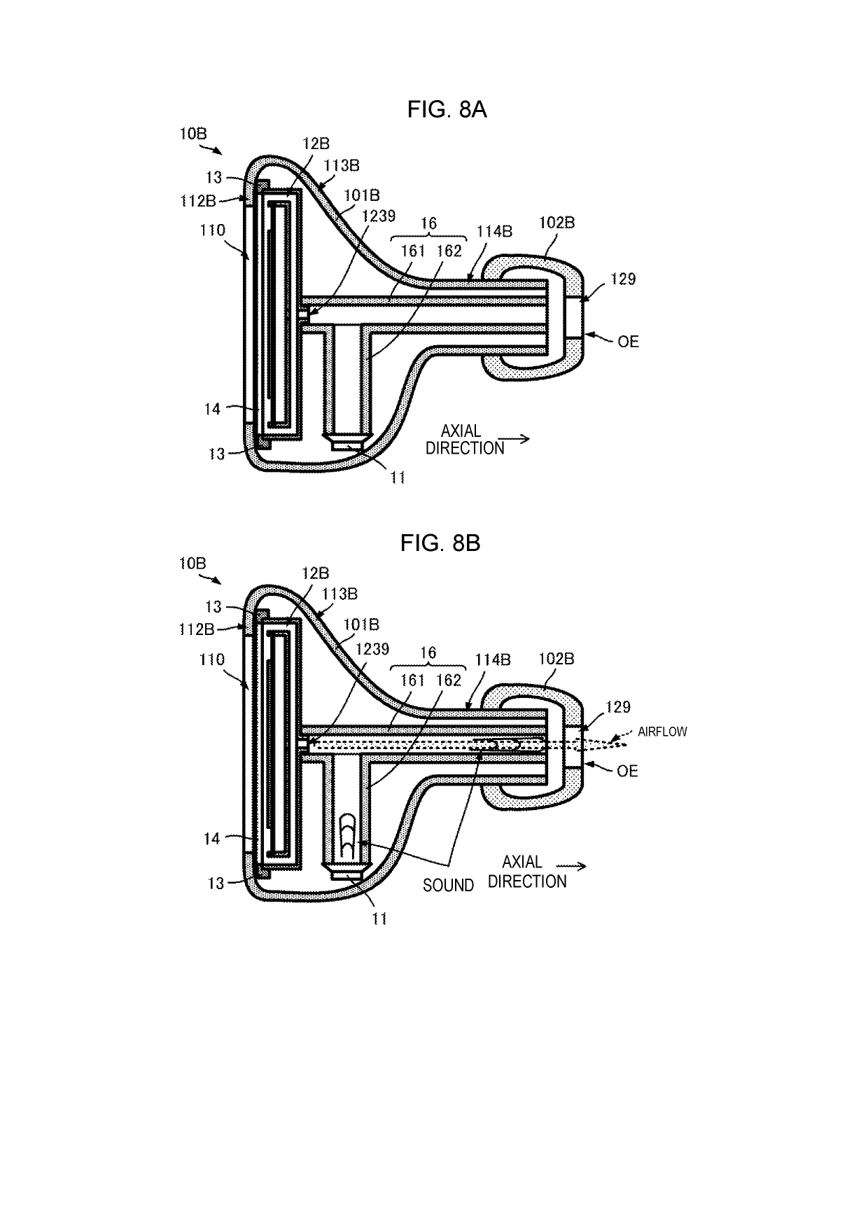

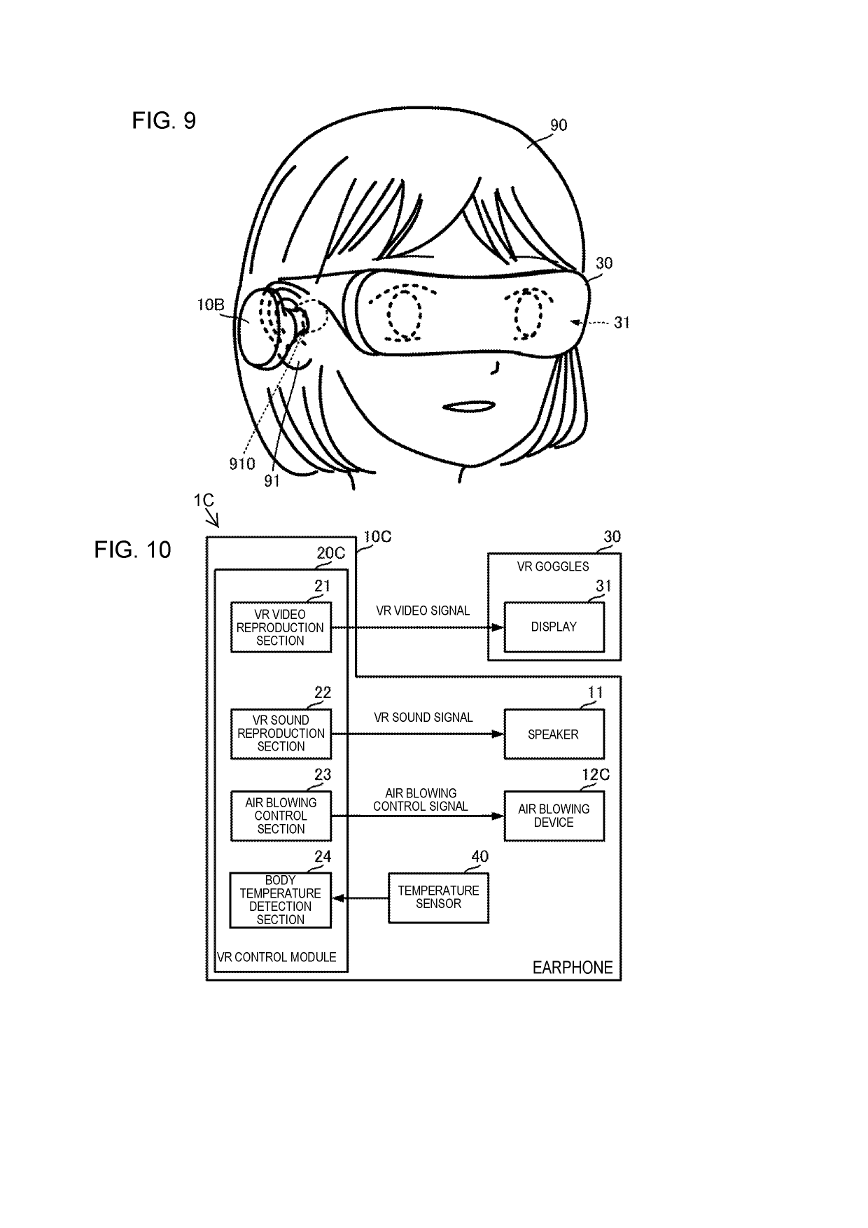

A VR apparatus according to a third embodiment of the present disclosure is described with reference to the drawings. FIG. 8A is a side cross-sectional view depicting an example of a configuration of an earphone of the VR apparatus according to the third embodiment. FIG. 8B is a side cross-sectional view depicting an example of flows of airflow and sound. FIG. 9 is a diagram depicting an example of how the VR apparatus according to the third embodiment is worn.

As depicted in FIGS. 8A and 8B, a VR apparatus 1B according to the third embodiment is different from the VR apparatus 1A according to the second embodiment in that an earphone 10B includes a housing 101B having a different shape, an ear pad 102B having a different shape, and a flow passage tube 16. The other configuration of the VR apparatus 1B is the same as that of the VR apparatus 1A, and description of the same portions is omitted.

As depicted in FIGS. 8A and 8B, the earphone 10B of the VR apparatus 1B includes the housing 101B, the speaker 11, an air blowing device 12B, the first sound absorbing member 13, the second sound absorbing member 14, the flow passage tube 16, and the ear pad 102B. The fixing member of the disclosure of the present application is configured by the first sound absorbing member 13 and the second sound absorbing member 14.

The housing 101B has a shape having a bottom wall 112B, a side wall 113B, and a cylindrical portion 114B.

The bottom wall 112B is a substantially flat plate. The side wall 113B is erected along the outer periphery of the bottom wall 112B. The side wall 113B has a shape in which, in a direction orthogonal to the flat plate surface of the bottom wall 112B, the area of a cross-section parallel to the flat plate surface of the bottom wall 112B becomes smaller as the position of the cross-section becomes farther from the bottom wall 112B.

The cross-sectional area of an opening of the cylindrical portion 114B is smaller than the area of the flat plate surface of the bottom wall 112B. The cylindrical portion 114B is connected to an end portion of the side wall 113B on the side opposite to an end portion connected to the bottom wall 112B. An internal space of the cylindrical portion 114B communicates with an internal space surrounded by the bottom wall 112B and the side wall 113B. Further, these internal spaces form an internal space of the housing 101B. The housing 101B is composed of a material having rigidity that allows the shape to be kept constant, and is composed of, for example, a resin, a metal, or the like.

The ear pad 102B has an annular shape. The ear pad 102B is composed of a material having cushioning properties.

The ear pad 102B has the central space 129. The ear pad 102B is attached to the housing 101B such that the cylindrical portion 114B of the housing 101B is inserted into the central space 129. Thus, the internal space of the housing 101B and the central space 129 of the ear pad 102B communicate with each other. The opening surface of the ear pad 102B on the side opposite to the mounting surface to the housing 101B is the opening OE of the earphone 10B. The opening OE serves as a sound radiation surface and an airflow delivery surface as the earphone 10B.

The air blowing device 12B is configured with a piezoelectric pump similar to the air blowing device 12A. The air blowing device 12B is disposed such that the discharge port 1239 is substantially orthogonal to the axial direction of the housing 101B and is oriented toward the opening OE.

The first sound absorbing member 13 is composed of a material having low air permeability and high sound absorbing properties. The first sound absorbing member 13 has an annular shape.

The first sound absorbing member 13 is disposed on the bottom wall 112B side of the housing 101B relative to the air blowing device 12B. In other words, the first sound absorbing member 13 is disposed on the side opposite to the opening OE with the air blowing device 12B being a reference in the housing 101B.

The first sound absorbing member 13 abuts against the bottom wall 112B. A surface of the first sound absorbing member 13 on the side opposite to the bottom wall 112B abuts against the air blowing device 12B.

The second sound absorbing member 14 is disposed in a through-hole penetrating the first sound absorbing member 13. The second sound absorbing member 14 overlaps the opening 110 of the bottom wall 112B of the earphone 10B.

The second sound absorbing member 14 is composed of a material having high air permeability and sound absorbing properties. The second sound absorbing member 14 is composed of, for example, glass wool, steel wool, or the like.

By including the first sound absorbing member 13 and the second sound absorbing member 14, the earphone 10B can suppress leakage of undesired sound generated by the air blowing device 12B to the outside of the housing 101B. Further, by including the second sound absorbing member 14, the earphone 10B can ensure air supply to the air blowing device 12B while suppressing leakage of the above undesired sound.

That is, the first sound absorbing member 13 primarily implements absorption of undesired sound, and the second sound absorbing member 14 ensures air supply to the air blowing device 12B while assisting in absorbing undesired sound.

The speaker 11 is fixed to the housing 101B. The speaker 11 is disposed such that a sound radiation surface thereof faces the internal space of the housing 101B and is exposed to this internal space.

A main sound radiation direction of the speaker 11 is orthogonal to the axial direction of the housing 101B. The manner in which the speaker 11 is disposed is not limited thereto. Various manners, as appropriate, can be adopted for the manner in which the speaker 11 is disposed, depending on the shape of the flow passage tube 16.

The shape of the speaker 11 is not limited, but the speaker 11 may be as small as possible.

The flow passage tube 16 includes a first tube 161 and a second tube 162.

The first tube 161 has a shape extending in a direction parallel to the axial direction of the housing 101B. One open end of the first tube 161 is connected to the discharge port 1239 of the air blowing device 12B. The other open end of the first tube 161 is disposed in the vicinity of the opening OE of the earphone 10B. In plan view of the opening OE, the other open end of the first tube 161 overlaps the opening OE.

The second tube 162 has a shape extending in a direction orthogonal to the axial direction of the housing 101B. One open end of the second tube 162 is connected to an intermediate position on the first tube 161 in the extension direction of the first tube 161. Thus, an internal space of the second tube 162 communicates with an internal space of the first tube 161. The other open end of the second tube 162 abuts against or is close to the sound radiation surface of the speaker 11. In plan view of the sound radiation surface, the opening portion (the internal space of the second tube 162) of the other open end overlaps the sound radiation surface.

With such a configuration, as depicted in FIG. 8B, airflow discharged from the air blowing device 12B passes through the first tube 161, and directly reaches the opening OE to be delivered from the opening OE. Sound radiated from the speaker 11 propagates through the second tube 162 to the first tube 161, and is radiated from the opening OE through the first tube 161.

As depicted in FIG. 9, the earphone 10B of the VR apparatus 1B is worn on the user 90 so as to be inserted into the ear canal opening 910 of the ear 91 of the user 90. The earphone 10B is worn on the right ear, and although depiction is omitted, the earphone 10B is also worn on the left ear.

Thus, the earphone 10B can provide sound and airflow from the opening OE to the ear 91 of the user 90. The sound and the airflow enter the external auditory canal 911 through the ear canal opening 910. The sound and the airflow that have entered the external auditory canal 911 reach the eardrum 919.

Thus, the user 90 can hear the sound and can also experience the sensation of the airflow almost simultaneously. Accordingly, the VR apparatus 1B can provide a high sense of immersion to the user 90.

Further, in the earphone 10B, airflow that is discharged from the air blowing device 12B configured with the piezoelectric pump and has high directivity is directly delivered from the opening OE. Thus, the earphone 10B can deliver the airflow efficiently and effectively.

In addition, sound radiated from the speaker 11 does not have strong directivity and, for example, is transmitted to the opening OE with substantially low loss while being reflected by an inner wall surface also inside the flow passage tube 16. Thus, the earphone 10B can radiate the sound with low loss.

On the other hand, for example, when the positional relationship between the air blowing device 12B and the speaker 11 is reversed, sound from the speaker 11 is directly radiated from the opening OE, but air (air current) discharged from the air blowing device 12B is likely to cause, for example, turbulence or the like inside the flow passage tube 16 and tends to incur loss.

As described above, by including the configuration of the earphone 10B, the VR apparatus 1B can deliver airflow to the ear canal opening 910 of the user 90 efficiently and effectively.

### FOURTH EMBODIMENT

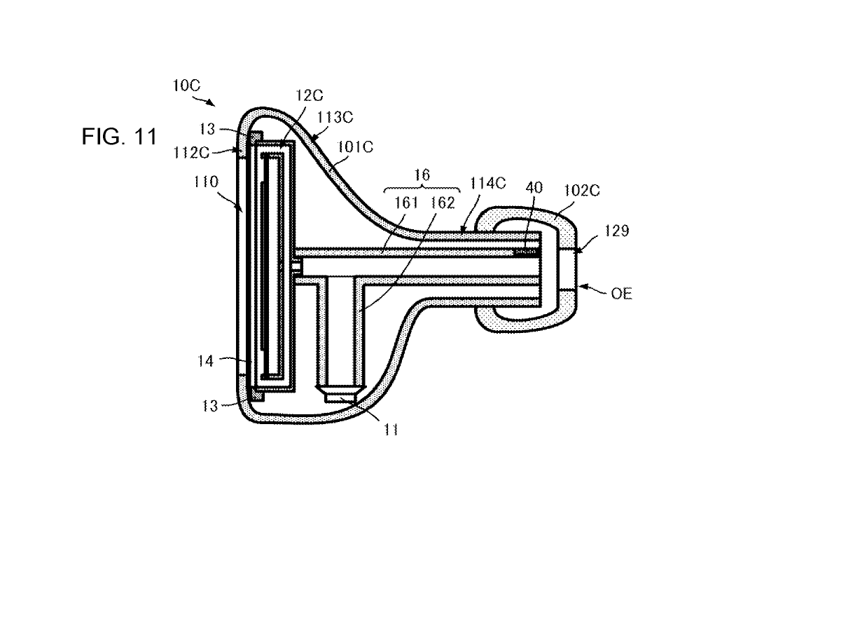

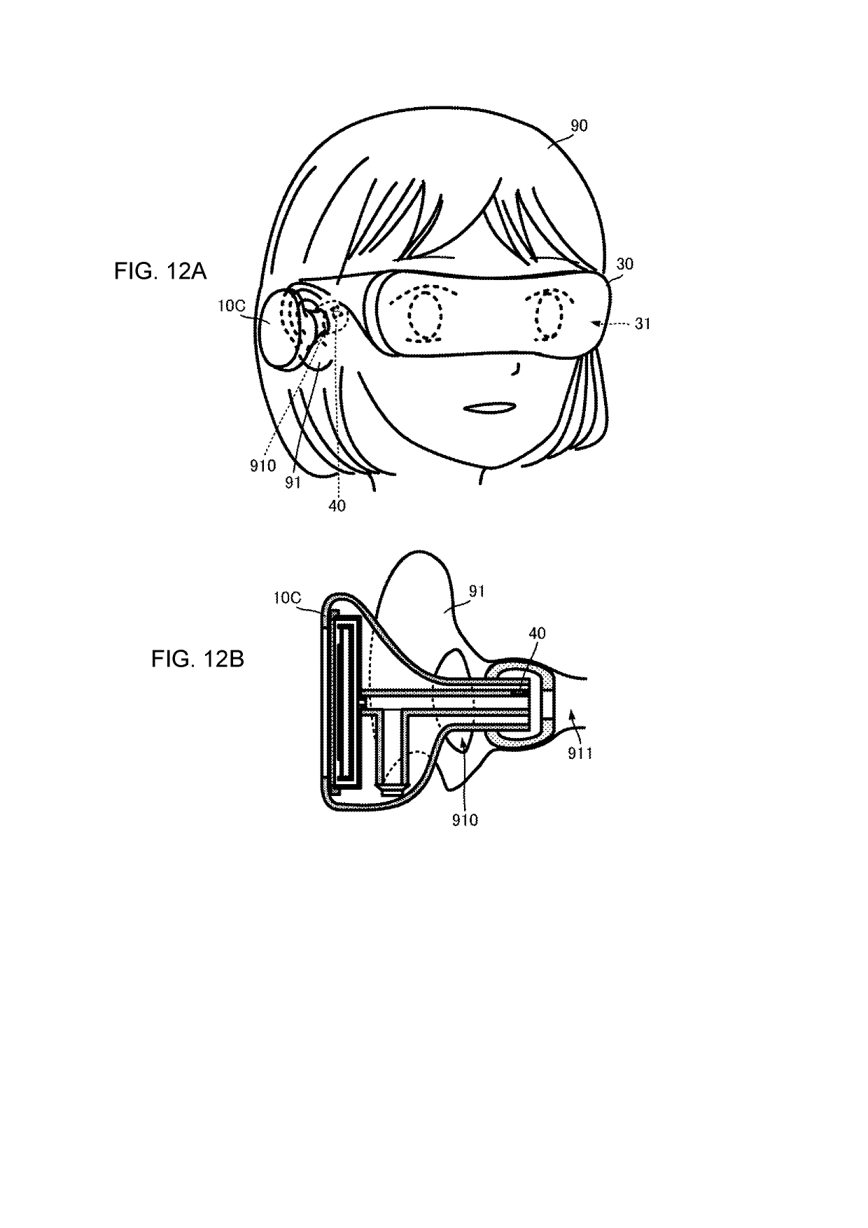

A VR apparatus according to a fourth embodiment of the present disclosure is described with reference to the drawings. FIG. 10 is a functional block diagram of the VR apparatus according to the fourth embodiment. In FIG. 10, a speaker for the right channel and a speaker for the left channel are collectively indicated as the speaker 11, and an air blowing device for the right ear and an air blowing device for the left ear are collectively indicated as an air blowing device 12C. FIG. 11 is a side cross-sectional view depicting an example of a configuration of an earphone of the VR apparatus according to the fourth embodiment. FIG. 12A is a perspective view depicting an example of how the VR apparatus according to the fourth embodiment is worn. FIG. 12B is an enlarged cross-sectional view of a vicinity of a wearing position.

As depicted in FIGS. 10, 11, 12A, and 12B, a VR apparatus 1C according to the fourth embodiment is different from the VR apparatus 1B according to the third embodiment in that an earphone 10C is provided with a temperature sensor 40. The other configuration of the VR apparatus 1C is the same as that of the VR apparatus 1B, and description of the same portions is omitted.

The earphone 10C includes the temperature sensor 40. The temperature sensor 40 is, for example, a chip-type thermistor. The temperature sensor 40 is disposed in or on the first tube 161 of the flow passage tube 16 of the earphone 10C. The temperature sensor 40 may be disposed at a position near the tip of the first tube 161. The tip of the first tube 161 is the end on the side opposite to the side connected to the air blowing device 12C and on the side closer to the opening OE of the earphone 10C. The temperature sensor 40 may be disposed at the tip of a cylindrical portion 114C of a housing 101C, or may be disposed in or on an ear pad 102C.

In such a configuration, when the user 90 wears the earphone 10C, the temperature sensor 40 is disposed inside the ear canal opening 910, as depicted in FIGS. 12A and 12B. Thus, the temperature sensor 40 can detect the body temperature of the user 90 with high accuracy.

The temperature sensor 40 outputs a temperature detection signal based on the body temperature to a VR control module 20C.

The VR control module 20C includes a body temperature detection section 24. The body temperature detection section 24 detects the body temperature on the basis of the temperature detection signal. The air blowing control section 23 generates and outputs the air blowing control signal on the basis of the body temperature. For example, when detecting that the detected body temperature is greater than or equal to a threshold for cooling, the air blowing control section 23 generates the air blowing control signal and outputs it to the air blowing device 12C.

Thus, the VR control module 20C can cool the user 90 by blowing air when the body temperature of the user 90 becomes high. At this time, the air blowing device 12C delivers airflow to the ear canal opening 910 and thus can effectively cool the user 90.

### FIFTH EMBODIMENT

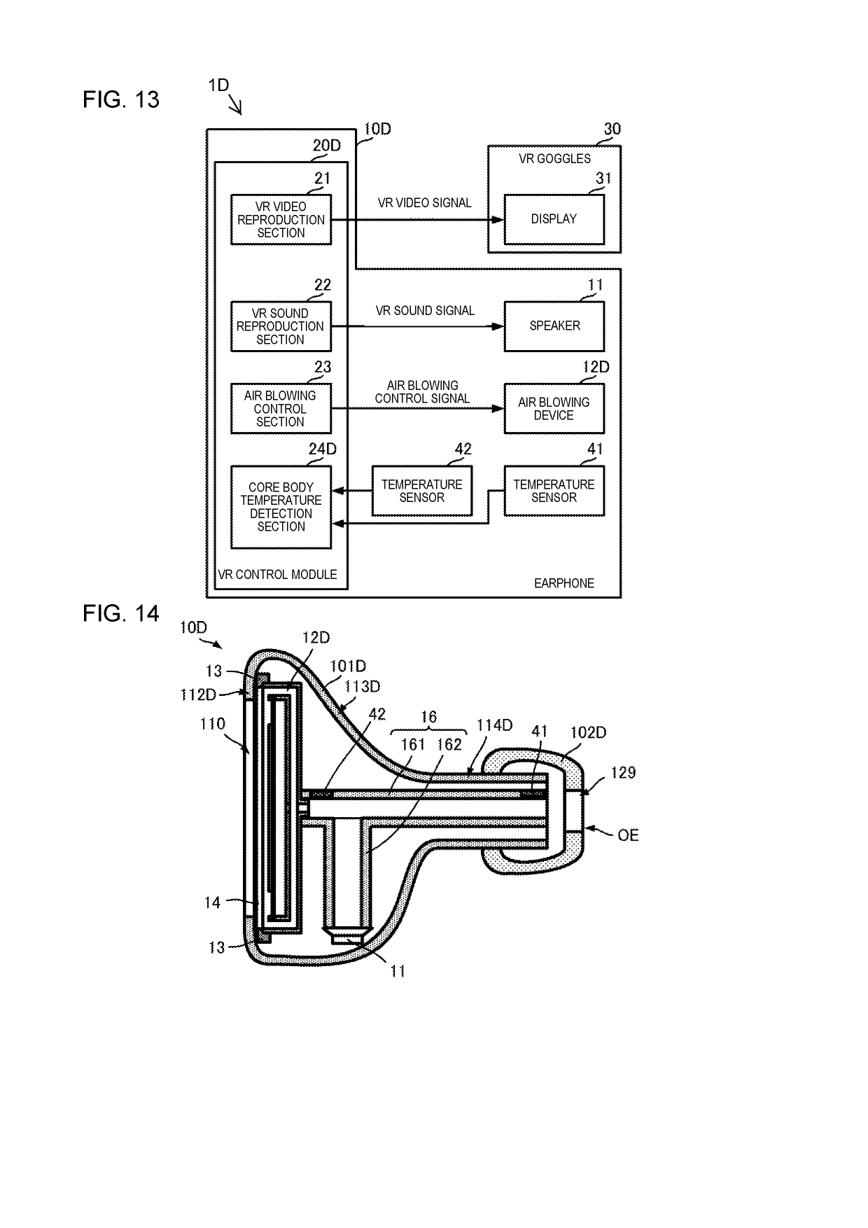

A VR apparatus according to a fifth embodiment of the present disclosure is described with reference to the drawings. FIG. 13 is a functional block diagram of the VR apparatus according to the fifth embodiment. In FIG. 13, a speaker for the right channel and a speaker for the left channel are collectively indicated as the speaker 11, and an air blowing device for the right ear and an air blowing device for the left ear are collectively indicated as an air blowing device 12D. FIG. 14 is a side cross-sectional view depicting an example of a configuration of an earphone of the VR apparatus according to the fifth embodiment.

As depicted in FIGS. 13 and 14, a VR apparatus 1D is different from the VR apparatus 1C according to the fourth embodiment in that an earphone 10D is provided with a temperature sensor 41 and a temperature sensor 42. The other configuration of the VR apparatus 1D is the same as that of the VR apparatus 1C, and description of the same portions is omitted.

The earphone 10D includes the temperature sensor 41 and the temperature sensor 42. The temperature sensor 41 and the temperature sensor 42 are, for example, chip-type thermistors similar to the temperature sensor 40. The temperature sensor 41 and the temperature sensor 42 are disposed in or on the first tube 161 of the flow passage tube 16 of the earphone 10D. The temperature sensor 41 is disposed in the vicinity of the other open end of the first tube 161. The temperature sensor 42 is disposed in the vicinity of the one open end of the first tube 161.

In such a configuration, when the user 90 wears the earphone 10D, the temperature sensor 41 is disposed inside the ear canal opening 910, and the temperature sensor 42 is disposed outside the ear canal opening 910 at a position distant from the ear canal opening 910. The temperature sensor 41 and the temperature sensor 42 output the temperature detection signals to a VR control module 20D.

The VR control module 20D includes a body temperature detection section 24D. The body temperature detection section 24D detects the core body temperature of the user 90 on the basis of the temperature detection signal of the temperature sensor 41 and the temperature detection signal of the temperature sensor 42. The air blowing control section 23 generates and outputs the air blowing control signal on the basis of the core body temperature. For example, when detecting that the core body temperature is greater than or equal to a threshold for cooling, the air blowing control section 23 generates the air blowing control signal and outputs it to the air blowing device 12D.

Thus, the VR control module 20D can cool the user 90 by blowing air when the core body temperature of the user 90 becomes high. At this time, the air blowing device 12D delivers airflow to the ear canal opening 910 and thus can effectively cool the user 90.

### SIXTH EMBODIMENT

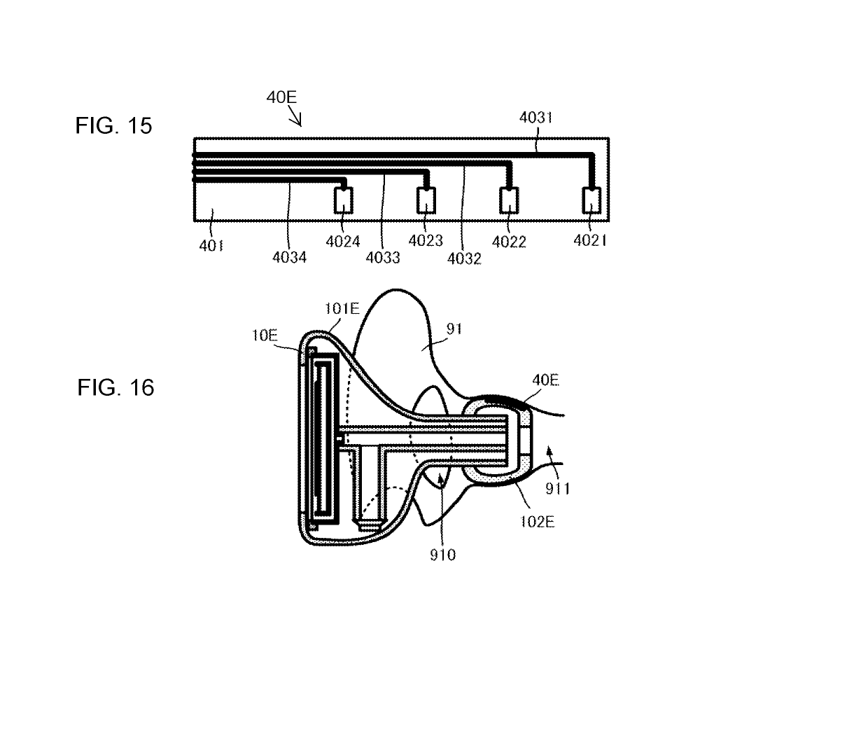

A VR apparatus according to a sixth embodiment of the present disclosure is described with reference to the drawings. FIG. 15 is a schematic configuration diagram of a temperature sensor 40E of the VR apparatus according to the sixth embodiment. FIG. 16 is an enlarged cross-sectional view of a vicinity of a wearing position of the VR apparatus according to the sixth embodiment.

The VR apparatus according to the sixth embodiment is different from the VR apparatus 1C according to the fourth embodiment in that it includes the temperature sensor 40E. The other configuration of the VR apparatus according to the sixth embodiment is the same as that of the VR apparatus 1C according to the fourth embodiment, and description of the same portions is omitted.

As depicted in FIG. 15, the temperature sensor 40E includes a base portion 401, a plurality of thermistors 4021 to 4024, and a plurality of wiring patterns 4031 to 4034. The base portion 401 is formed of an insulating film that has flexibility and is deformable. The base portion 401 has an elongated shape.

The thermistors 4021 to 4024 are disposed at intervals in the extension direction of the base portion 401. The wiring patterns 4031 to 4034 are connected to the thermistors 4021 to 4024, respectively. The wiring patterns 4031 to 4034 are connected to the thermistors 4021 to 4024 in a connection manner that enables extraction of temperature detection signals of the thermistors 4021 to 4024, although depiction is omitted.

The temperature sensor 40E is disposed in or on an ear pad 102E. Thus, the thermistors 4021 to 4024 of the temperature sensor 40E abut against or are brought close to a wall surface of the external auditory canal 911. Accordingly, the temperature detection signals of the thermistors 4021 to 4024 reflect the temperature of the user 90 with higher accuracy.

The temperature detection signals of the thermistors 4021 to 4024 are inputted to the body temperature detection section of the VR apparatus. The body temperature detection section detects the body temperature of the user 90 on the basis of the temperature detection signals of the thermistors 4021 to 4024.

In this manner, the VR apparatus according to the sixth embodiment can achieve operation and effects similar to those of the VR apparatus 1C according to the fourth embodiment. Further, the VR apparatus according to the sixth embodiment can detect the body temperature with high accuracy, and thus execute air blowing control based on the body temperature of the user 90 with high accuracy.

### Example of Activation Circuit of Air Blowing Control Section

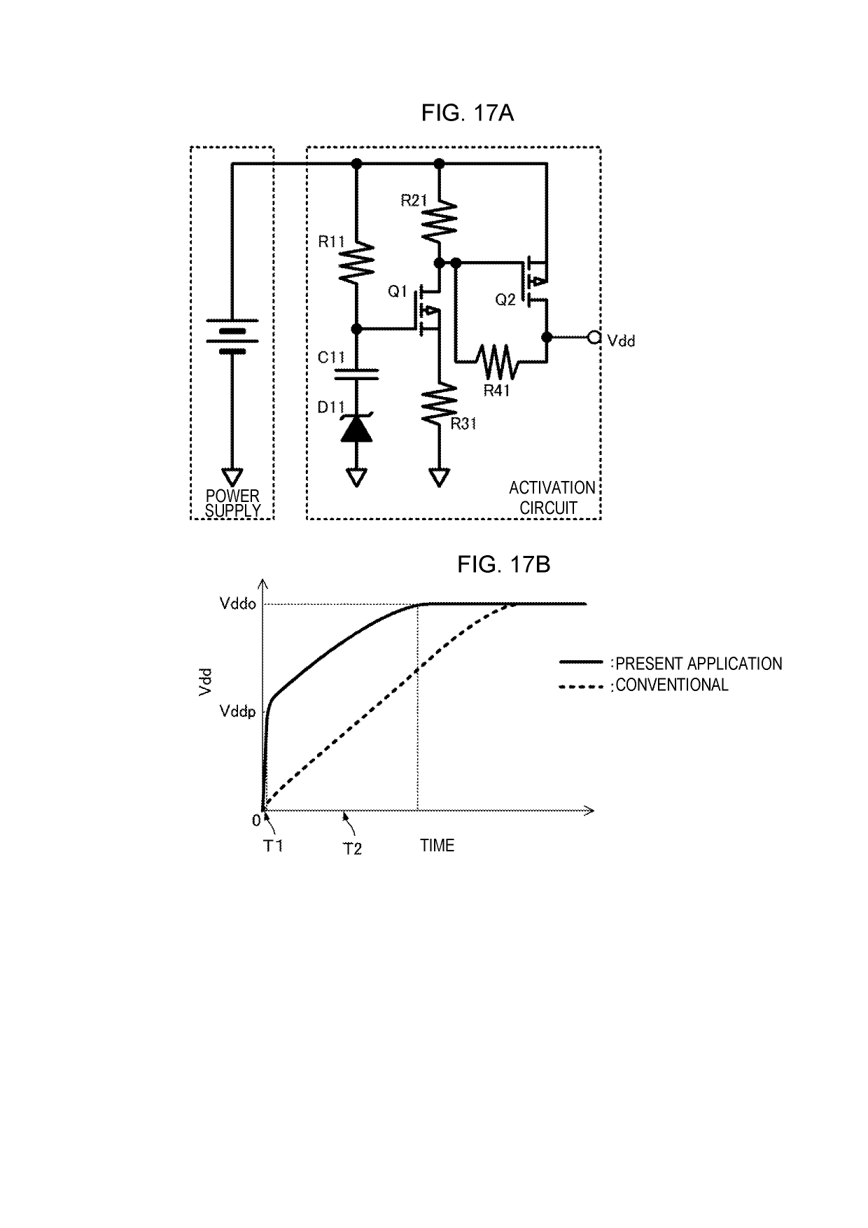

FIG. 17A is a circuit diagram depicting an example of an activation circuit of the air blowing control section. FIG. 17B depicts an output voltage characteristic of the activation circuit. The horizontal axis in FIG. 17B indicates an elapsed time from the start of driving (activation), and the vertical axis indicates an output voltage. This output voltage is the voltage applied to the air blowing device.

This activation circuit is suitable for a mode in which a piezoelectric pump is used as the air blowing device.

As depicted in FIG. 17A, the activation circuit includes a plurality of switching elements Q1 and Q2, a plurality of resistors R11, R21, R31, and R41, a capacitor C11, and a Zener diode D11.

A series circuit of the resistor R11, the capacitor C11, and the Zener diode D11 is connected between the positive electrode and the negative electrode of a DC power supply. The gate terminal of the switching element Q1 is connected to a node between the resistor R11 and the capacitor C11.

The resistor R21 is connected to the positive electrode of the DC power supply. The drain terminal of the switching element Q1 is connected to the resistor R21. The resistor R31 is connected to the source terminal of the switching element Q1. The resistor R31 is connected to the negative electrode of the DC power supply. The gate terminal of the switching element Q2 is connected to a node between the resistor R21 and the drain terminal of the switching element Q1. The drain terminal of the switching element Q2 is connected to the positive electrode of the DC power supply. The source terminal of the switching element Q2 is connected to an output terminal of the activation circuit. The source terminal of the switching element Q2 is connected to the gate terminal of the switching element Q2 through the resistor R41.

As depicted in FIG. 17B, the activation circuit having such a configuration can execute slope voltage control in which a voltage change rate in a voltage (activation voltage) at the start of generation of a supply voltage Vdd is set to a first stage and a second stage and the voltage change rate of the second stage is set lower than that of the first stage.

Thus, the activation circuit can suppress undesired power consumption at the time of activation, and increase the efficiency of power supply to the air blowing device configured with the piezoelectric pump.

The activation circuit is not limited to that shown as the example, and may be a circuit that controls the supply voltage Vdd by an MCU.

### Example of Drive Circuit of Air Blowing Control Section

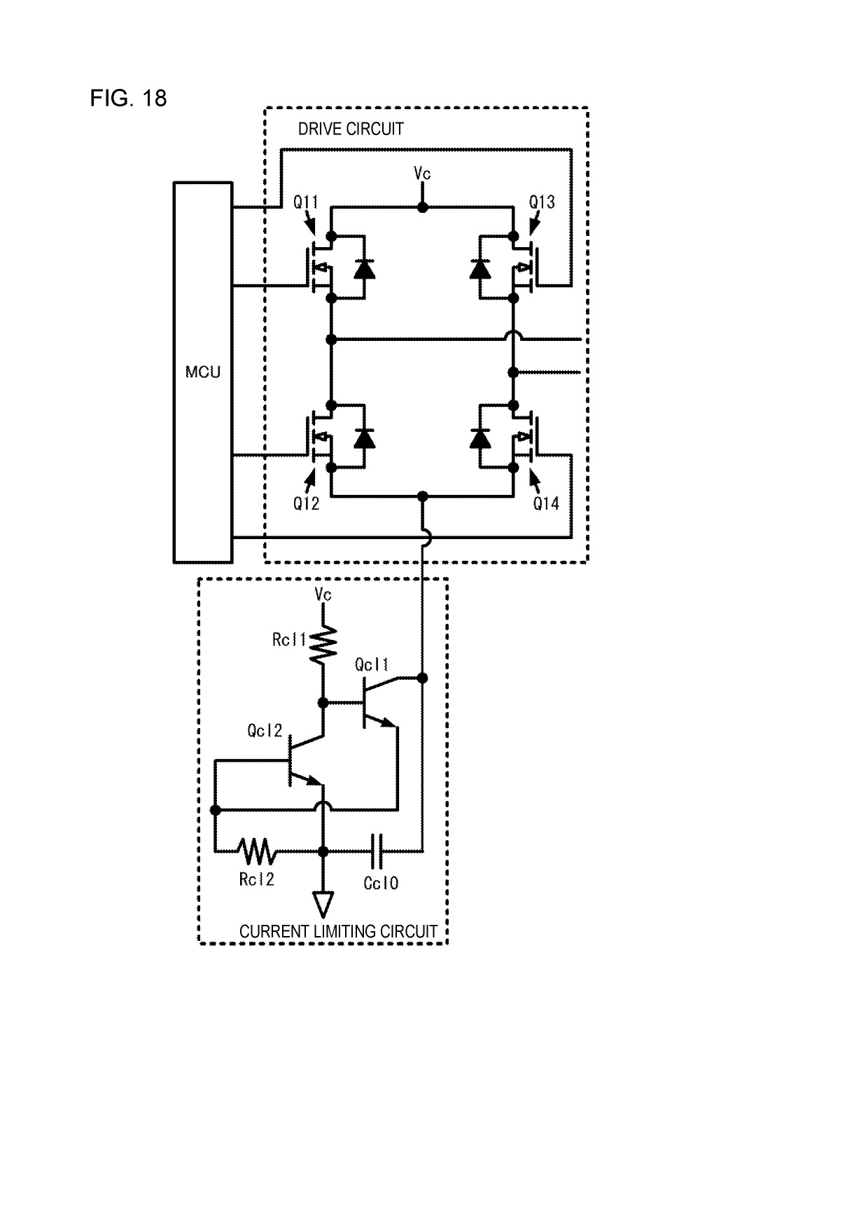

FIG. 18 is a circuit diagram depicting an example of a drive circuit of the air blowing control section. This drive circuit is suitable for a mode in which a piezoelectric pump is used as the air blowing device.

As depicted in FIG. 18, the drive circuit of a second example includes an H-bridge circuit controlled by an MCU and a current limiting circuit. The MCU is connected to the H-bridge circuit and outputs a PWM signal having appropriate frequency and duty ratio. The H-bridge circuit includes a plurality of switching elements Q11 to Q14. The respective gate terminals of the switching elements Q11 to Q14 are connected to the MCU. The drain terminal of the switching element Q11 and the drain terminal of the switching element Q13 are connected, and a drive voltage Vc is applied thereto. The source terminal of the switching element Q11 is connected to the drain terminal of the switching element Q12. The source terminal of the switching element Q13 is connected to the drain terminal of the switching element Q14. The source terminal of the switching element Q12 and the source terminal of the switching element Q14 are connected, and this connection node is connected to the current limiting circuit. The connection node between the source terminal of the switching element Q11 and the drain terminal of the switching element Q12 and the connection node between the source terminal of the switching element Q13 and the drain terminal of the switching element Q14 serve as output terminals of the drive circuit.

The current limiting circuit includes a transistor Qcl1, a transistor Qcl2, a resistor Rcl1, a resistor Rcl2, and a capacitor Ccl0.

The drive voltage Vc is applied to the base terminal of the transistor Qcl1 through the resistor Rcl1. This drive voltage Vc is the supply voltage Vdd indicated in FIG. 17A. The base terminal of the transistor Qcl1 is connected to the collector terminal of the transistor Qcl2. The Emitter terminal of the transistor Qcl2 is connected to a reference potential.

The collector terminal of the transistor Qcl1 is connected to the reference potential through the capacitor Ccl0. Further, the collector terminal of the transistor Qcl1 is connected to the connection node between the source terminal of the switching element Q12 and the source terminal of the switching element Q14.

The Emitter terminal of the transistor Qcl1 is connected to the base terminal of the transistor Qcl2. The connection node between the base terminal of the transistor Qcl2 and the Emitter terminal of the transistor Qcl1 is connected to the reference potential through the resistor Rcl2.

With such a configuration, the drive circuit and the current limiting circuit can set the drive voltage such that the air blowing device is driven for an airflow volume giving optimal power consumption and can suppress supply of an undesired current to the air blowing device. Thus, the drive circuit and the current limiting circuit can optimize the power consumption of the air blowing device configured with the piezoelectric pump, and increase the air blowing efficiency.

The drive circuit is not limited to that shown as the example, and may employ another circuit configuration, such as one configured with a linear amplifier.

The configurations of the above respective embodiments can be combined as appropriate, and operation and effects corresponding to each combination can be achieved.

<1> A VR apparatus comprising: a housing having a first opening capable of communicating with an ear canal opening of a user; an air blowing device that is provided in the housing and generates airflow to be delivered from the first opening to outside of the housing; a speaker that is provided in the housing and generates sound to be delivered from the first opening to the outside of the housing; and a VR control module configured to, on a basis of virtual reality provided to the user, output an air blowing control signal to the air blowing device and output a sound radiation control signal to the speaker in such a manner as to synchronize a generation timing of the sound and a generation timing of the airflow in time.

<2> The VR apparatus according to <1>, wherein the air blowing device is a piezoelectric pump using a piezoelectric element.

<3> The VR apparatus according to <1> or <2>, further comprising: a temperature sensor that detects a body temperature of the user, wherein the VR control module is configured to output the air blowing control signal with reference to the body temperature detected by the temperature sensor.

<4> The VR apparatus according to <3>, wherein the temperature sensor detects a core body temperature as the body temperature.

<5> The VR apparatus according to <3> or <4>, wherein the temperature sensor includes a base portion that is deformable.

<6> The VR apparatus according to any one of <1> to <5>, wherein the air blowing device includes a drive circuit that generates a drive signal of piezoelectric pump on a basis of the air blowing control signal, and the drive circuit executes slope voltage control in which a voltage change rate is set to a first stage and a second stage at a start of generation of the drive signal and the voltage change rate of the second stage is set lower than the voltage change rate of the first stage.

<7> The VR apparatus according to any one of <1> to <5>, wherein the air blowing device includes a drive circuit that generates a drive signal of piezoelectric pump on a basis of the air blowing control signal, and the drive circuit includes a current limiting circuit that limits an output current.

<8> The VR apparatus according to any one of <1> to <7>, wherein the VR control module is provided in the housing.

<9> The VR apparatus according to any one of <1> to <8>, further comprising: a display that displays video based on the virtual reality provided to the user.

<10> The VR apparatus according to any one of <1> to <9>, further comprising: a fixing member disposed on a side opposite to the first opening with the air blowing device being a reference in the housing, the fixing member fixing the air blowing device to the housing, wherein the fixing member includes a first sound absorbing member in which air permeability is low and sound absorbing properties are high, and a second sound absorbing member that is disposed in a through-hole penetrating the first sound absorbing member and has the sound absorbing properties, the air permeability of the second sound absorbing member being high.

<11> The VR apparatus according to any one of <1> to <10>, wherein the VR apparatus has an auxiliary air-discharge hole formed between the air blowing device and the first opening in the housing.

<12> The VR apparatus according to any one of <1> to <11>, wherein a discharge port of the air blowing device is disposed at a position overlapping the first opening when the first opening is viewed from the outside.

<13> The VR apparatus according to any one of <1> to <12>, wherein a front direction of the speaker is a direction different from a direction connecting a position of the speaker and the first opening.

<14> The VR apparatus according to any one of <1> to <13>, wherein the VR control module is configured to output the air blowing control signal at a timing of giving coolness to the user, a timing of giving a surprise to the user, or a timing of causing the user to have a sense of fear in the virtual reality.

1, 1A, 1B, 1C, 1D VR apparatuses

10, 10A headphones

10B, 10C, 10D earphones

11, 11L, 11R speakers

12, 12A, 12B, 12C, 12D, 12L, 12R air blowing devices

13 first sound absorbing member

14 second sound absorbing member

15 holding member

16 flow passage tube

20, 20C, 20D VR control modules

21 VR video reproduction section

22 VR sound reproduction section

23 air blowing control section

24 body temperature detection section

24D body temperature detection section

30 VR goggles

31 display

40, 40E, 41, 42 temperature sensors

90 user

91 ear

101, 101B, 101D housings

102, 102B, 102E ear pads

109 through-hole

110 opening

112, 112B bottom walls

113, 113B side walls

114B, 114D cylindrical portions

119 auxiliary air-discharge hole

121 housing

122 fan

123 outer housing

124 inner housing

125 diaphragm

126 piezoelectric element

1290 pump chamber

129 central space

161 first tube

162 second tube

401 base portion

910 ear canal opening

911 external auditory canal

919 eardrum

1230 flow passage

1239 discharge port

1249 through-hole

4021 to 4024 thermistors

4031 to 4034 wiring patterns

1. A VR apparatus comprising:
a housing having a first opening configured to communicate with an ear canal opening of a user;
an air blowing device provided in the housing and configured to generate airflow to be delivered from the first opening to outside of the housing;
a speaker provided in the housing and configured to generate sound to be delivered from the first opening to the outside of the housing; and
a VR control module configured to, on a basis of virtual reality provided to the user, output an air blowing control signal to the air blowing device and output a sound radiation control signal to the speaker in such a manner as to synchronize a generation timing of the sound and a generation timing of the airflow in time.

2. The VR apparatus according to claim 1, wherein
the air blowing device is a piezoelectric pump using a piezoelectric element.

3. The VR apparatus according to claim 1, further comprising:
a temperature sensor configured to detect a body temperature of the user, wherein
the VR control module is configured to output the air blowing control signal with reference to the body temperature detected by the temperature sensor.

4. The VR apparatus according to claim 3, wherein
the temperature sensor is configured to detect a core body temperature as the body temperature.

5. The VR apparatus according to claim 3, wherein
the temperature sensor includes a deformable base portion.

6. The VR apparatus according to claim 1, wherein
the air blowing device includes a drive circuit configured to generate a drive signal of piezoelectric pump on a basis of the air blowing control signal, and
the drive circuit is configured to execute slope voltage control in which a voltage change rate is set to a first stage and a second stage at a start of generation of the drive signal and the voltage change rate of the second stage is set lower than the voltage change rate of the first stage.

7. The VR apparatus according to claim 1, wherein
the air blowing device includes a drive circuit configured to generate a drive signal of piezoelectric pump on a basis of the air blowing control signal, and
the drive circuit includes a current limiting circuit that limits an output current.

8. The VR apparatus according to claim 1, wherein
the VR control module is provided in the housing.

9. The VR apparatus according to claim 1, further comprising:
a display configured to display video based on the virtual reality provided to the user.

10. The VR apparatus according to claim 1, further comprising:
a fixing member disposed on a side opposite to the first opening with the air blowing device being a reference in the housing, the fixing member fixing the air blowing device to the housing, wherein
the fixing member includes
a first sound absorbing member having low air permeability and high sound absorbing properties, and
a second sound absorbing member disposed in a through-hole penetrating the first sound absorbing member and having the sound absorbing properties, the air permeability of the second sound absorbing member being high.

11. The VR apparatus according to claim 1, wherein
the VR apparatus has an auxiliary air-discharge hole provided between the air blowing device and the first opening in the housing.

12. The VR apparatus according to claim 1, wherein
a discharge port of the air blowing device is disposed at a position overlapping the first opening when the first opening is viewed from the outside.

13. The VR apparatus according to claim 1, wherein
a front direction of the speaker is a direction different from a direction connecting a position of the speaker and the first opening.

14. The VR apparatus according to claim 1, wherein
the VR control module is configured to output the air blowing control signal at a timing of giving coolness to the user, a timing of giving a surprise to the user, or a timing of causing the user to have a sense of fear in the virtual reality.

15. The VR apparatus according to claim 2, further comprising:
a temperature sensor configured to detect a body temperature of the user, wherein
the VR control module is configured to output the air blowing control signal with reference to the body temperature detected by the temperature sensor.

16. The VR apparatus according to claim 4, wherein
the temperature sensor includes a deformable base portion.

17. The VR apparatus according to claim 2, wherein
the air blowing device includes a drive circuit configured to generate a drive signal of piezoelectric pump on a basis of the air blowing control signal, and
the drive circuit is configured to execute slope voltage control in which a voltage change rate is set to a first stage and a second stage at a start of generation of the drive signal and the voltage change rate of the second stage is set lower than the voltage change rate of the first stage.

18. The VR apparatus according to claim 3, wherein
the air blowing device includes a drive circuit configured to generate a drive signal of piezoelectric pump on a basis of the air blowing control signal, and
the drive circuit is configured to execute slope voltage control in which a voltage change rate is set to a first stage and a second stage at a start of generation of the drive signal and the voltage change rate of the second stage is set lower than the voltage change rate of the first stage.

19. The VR apparatus according to claim 4, wherein
the air blowing device includes a drive circuit configured to generate a drive signal of piezoelectric pump on a basis of the air blowing control signal, and
the drive circuit is configured to execute slope voltage control in which a voltage change rate is set to a first stage and a second stage at a start of generation of the drive signal and the voltage change rate of the second stage is set lower than the voltage change rate of the first stage.

20. The VR apparatus according to claim 5, wherein
the air blowing device includes a drive circuit configured to generate a drive signal of piezoelectric pump on a basis of the air blowing control signal, and
the drive circuit is configured to execute slope voltage control in which a voltage change rate is set to a first stage and a second stage at a start of generation of the drive signal and the voltage change rate of the second stage is set lower than the voltage change rate of the first stage.
